(19) United States
(12) Patent Application Publication
(10) Pub. No.: US 2016/0015625 A1
(43) Pub. Date: Jan. 21, 2016

(54) Method of Applying Organic Virgin Coconut Oil to Eyelashes and Eyebrows

(71) Applicant: Mary T. Stone, Raleigh, NC (US)

(72) Inventor: Mary T. Stone, Raleigh, NC (US)

(21) Appl. No.: 14/627,619

(22) Filed: Feb. 20, 2015

Related U.S. Application Data
(60) Provisional application No. 62/011,327, filed on Jun. 12, 2014.

(51) Int. Cl. A61K 8/97; A61Q 5/12; A45D 34/04

(52) U.S. Cl. CPC A61K 8/97; A45D 34/046; A61Q 5/12; A61K 2800/87

(57) ABSTRACT

A method for applying undiluted 100% Organic Virgin Coconut Oil to the eyelashes and/or eyebrows of a user via a brush is described. The brush employed is preferably equipped with a series of off-set bristles configured to be soft on the lashes, yet firm enough to adequately apply the coconut oil to the lashes and distribute the coconut oil evenly across all lashes. Via this method, the organic virgin coconut oil is applied to the lashes such to ensure that each lash or brow is effectively moisturized in order to enhance the life and aesthetic quality of the eye lashes and/or eyebrows.

### CONTINUITY

This application is a non-provisional application of provisional application No. 62/011,327, filed on Jun. 12, 2014, and priority is claimed thereto.

### FIELD OF THE PRESENT INVENTION

The present invention relates to cosmetic application methods, and more specifically, a means by which 100% organic virgin coconut oil may be applied to the eyelashes and eyebrows of a user such to appropriately and adequately condition the eyebrows and eyelashes of the user.

### BACKGROUND OF THE PRESENT INVENTION

It is commonly known that coconut oil is used as an ingredient in many cosmetic, skin care, cooking, food, and is found in many other products as well. As an example, many eye mascaras use coconut oil as a minor ingredient combined with many other products. Coconut oil is known to have many natural health benefits. Unfortunately coconut oil is rarely ever used as the sole or primary ingredient of a product, as high quality coconut oil can be prohibitively expensive to use exclusively without filler. Mascara companies are primarily driven by appearance. Conversely, the use of a pure coconut oil primarily benefits the health of the skin and lashes, as well as the appearance of the lashes over time. No products are found to employ an easy-to-use, travel-size tube in combination with virgin coconut oil in order to facilitate the application of the virgin coconut oil effectively.

Even without a conventional means of application, creative users of coconut oil have found alternative means of applying coconut oil to their lashes. Presently, it has been found that user's of 100% organic coconut oil to moisturize and condition eye lashes and eye brows utilize devices such as Q-tips, used mascara brushes, fingers, and other similar objects to apply coconut oil, often poorly, to the lashes and eyebrows after dipping the object into a jar of coconut oil. Clearly, this is a poor means of applying coconut oil evenly to the lashes or eye brows.

Additionally, it is known that non-organic coconut oil is frequently fertilized with harmful chemicals. This is unfortunate for most uses of coconut oil, as the chemicals may lead to poor health over time. Organic Virgin Coconut Oil is from coconut palm trees (cocos nucifera) that are fertilized with natural products, and are not fertilized with chemicals. Ideally, Organic Virgin Coconut Oil is extracted from fresh coconut meat. During processing, no chemicals are ever used. It is preferable to use such a virgin organic coconut oil in products designed for lashes, however it is not used by anyone in the market presently.

Thus, there is a need for a product that is 100% organic virgin coconut oil in order to maximize the benefits potentially possible through coconut oil, and that includes a brush, tool, and method of coconut oil application that facilitates and expedites the coconut oil application process.

### SUMMARY OF THE PRESENT INVENTION

The present invention is a method of application of a 100% organic virgin coconut oil which facilitates the even application and distribution of the organic virgin coconut oil to the eye lashes and/or eye brows of a user. The present invention employs a brush and a tube (or similarly shaped container), similar to those employed by conventional mascara tubes and applicators. The brush of the present invention preferably employs a series of bristles that are off-set in order to facilitate application of the organic virgin coconut oil to the eyelashes and/or eye brows of the user. The brush is preferably equipped with a grip that is easy to handle with dexterity while remaining comfortable, helping to prevent accidental dropping of the brush, and aiding even application of the organic virgin coconut oil to the target eye lashes and/or eyebrows.

The tube of the present invention is preferably equipped with a chamber which holds the organic virgin coconut oil securely. The tube is also equipped with a tip which is specially designed to wipe the brush of extraneous organic virgin coconut oil upon removal of the brush from the tube, similar to that of conventional mascara tubes. The primary difference between conventional mascara tubes and the tube of the present invention is that the tube employed by the present invention contains organic virgin coconut oil, and is equipped with a small brush housed within the tip, configured to facilitate the removal of any extra organic virgin coconut oil from the tube. It should be understood that the brush of the present invention is an applicator brush, and is preferably equipped with a conical or rounded tip. It is conventionally known that such a tip is best for ‘rolling’ a solution onto the eyelashes of a user.

### BRIEF DESCRIPTION OF THE DRAWINGS

FIG. 1 illustrates a side/top perspective view of the present invention including the brush, tube, and organic virgin coconut oil.

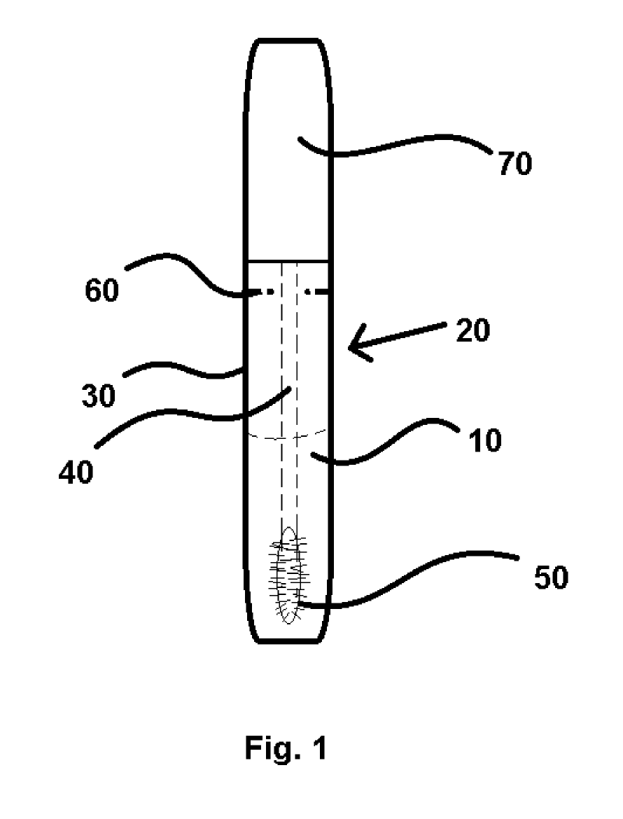

FIG. 2 exhibits the process of use of the present invention.

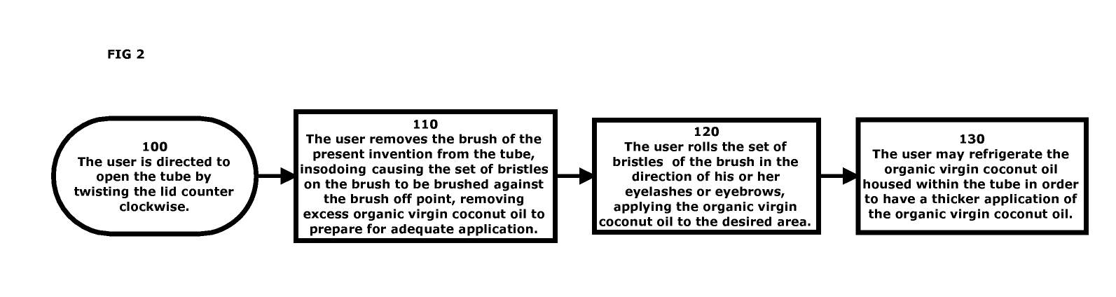

### DETAILED DESCRIPTION OF THE PREFERRED EMBODIMENT

The present invention is a method and system for the deliberate and exact application of an organic virgin coconut oil (10) to the eye lashes and eye brows of a user. The preferred embodiment of the present invention employs a tube (20), the tube (20) housing a container (30), and a brush (40). The brush (40) is equipped with a first end (170) and a second end (180).

The brush (40) of the present invention is preferably similar to a conventional mascara brush in that it is conical or rounded with a variety of bristles that are similar in length. The second end (180) of the brush (40) of the present invention is equipped with a set of bristles (50) which are preferably off-set from one another, and vary slightly in length. The set of bristles (50) of the present invention are configured to adhere slightly to the eyelashes or eyebrows of the user, while simultaneously providing a conduit for the eyelash or eyebrow to travel through during the procedure of application of the organic virgin coconut oil (10). The set of bristles (50) of the present invention are preferably arranged in a spiral-shaped pattern to form a conical or cylinder shaped tip at the second end of the brush (40).

Additionally, the first end (170) of the brush (40) is equipped with a cap (70). The cap (70) is preferably equipped with a handle (160) to facilitate handling the brush (40) without spilling the organic virgin coconut oil (10) and to maximize dexterity during the application process of the present invention. The bristles of the set of bristles (50) of the brush (40) of the present invention may vary in size from approximately 2 mm to 4 mm. The bristles near the tip of the brush (40) are preferably shorter than the remainder of the set of bristles (50). Thus, there is preferably a tapering of the bristles of the set of bristles (50) such that they vary in size of up to 3 mm, wherein the shortest bristles are near the tip of the brush (40), and the longer bristles of the set of bristles (50) are nearest the handle (160).

The container (30) housed within the tube (20) preferably contains a brush off point (60) in the preferred embodiment of the present invention. The brush off point (60) is ideally a small circumferential brush configured to ‘brush off’ excess organic virgin coconut oil (10) in order to ensure that the brush (40) is primed with an appropriate amount of organic virgin coconut oil (10) to be applied to a singular set of eyelashes or eye brows found above a single eye. The present invention may employ more than one instance of the brush off point (60), in order to ensure finer amounts of the organic virgin coconut oil (10) is available to be applied with a single removal of the brush (40) of the present invention. The brush (40) of the present invention is preferably integrated with a cap (70) of the present invention, in order to ensure that the tube (20) remains securely closed when not in use. The cap (70) of the present invention is equipped with handle (160). The handle (160) preferably has a contoured grip that may include a silicone or rubber-based grip lining to facilitate high dexterity when applying the organic virgin coconut oil (10) via the process of the present invention.

The method of application of the present invention in the preferred embodiment is as follows:

The user is directed to open the tube (20) of the present invention by twisting the cap (70) of the present invention counter clockwise. (100) Next, the user removes the brush (40) of the present invention from the tube (20), causing the set of bristles (50) on the brush (40) to be brushed against the brush off point (60), removing excess organic virgin coconut oil (10) to prepare for adequate application (110). Next, the user rolls the set of bristles (50) of the brush (40) in the direction of his or her eyelashes or eyebrows, applying the organic virgin coconut oil (10) to the desired area. (120). Given that the organic virgin coconut oil (10) is 100% pure, it is capable of being solid, or not firm at below 76 degrees Fahrenheit, and is found to be in a liquid form at any temperature above 76 degrees Fahrenheit. As such, the user may refrigerate the organic virgin coconut oil (10) housed within the tube (20) in order to have a thicker application of the organic virgin coconut oil (10) (130).

In colder climates, the user may be requested to heat up the tube (20) of the present invention in order to ensure that the organic virgin coconut oil (10) is in a liquid state to be applied to the lashes. (140). The organic virgin coconut oil (10) may be warmed slightly simply by grasping the container (30) with a hand, employing body heat to turn the organic virgin coconut oil (10) into a liquid form. Ideally, the container (30) of the present invention is preferably filled with organic virgin coconut oil (10) in liquid form. When the brush (40) of the present invention is pulled from the tube (20) via the handle (160), the brush off point (60) ensures the set of bristles (50) of the brush (40) are not clogged with an excess of the organic virgin coconut oil (10). (150). The user is therefore ensured that the process of application of the present invention delivers the moisturizing and conditioning liquid coconut oil to eye lashes and eye brows evenly and effectively.

In the event that the environment is colder than 76 degrees Fahrenheit, then the organic virgin coconut oil (10) on the brush (40) is pulled from the tube in solid form. During application, when the solid organic virgin coconut oil (10) touches or approaches the user's skin, it becomes a liquid, facilitating the moisturizing and conditioning to the eye lashes and eye brows.

It should be understood that the container (30) used in the process and method of application of the present invention may vary in size, shape, and color in accordance with availability. Similarly, the preferred size of the overall length of the cap (70) and brush (40) is approximately 12 mm. The brush (40) is preferably approximately ¼ to ⅓^{rd} the size of the overall length of the container (30) of the present invention.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A method for applying a solution containing 100% extra virgin olive oil to the eyelashes and eyebrows of a user comprising:
depositing 100% extra virgin olive oil within a container;
wherein said container is equipped with an opening, said opening has a diameter smaller than the circumference of said container;
wherein said container is equipped with a brush;
wherein said brush is equipped with a first end and a second end;
wherein the container the opening is equipped with a brush-off point having a circumference smaller than the second end of the brush;
wherein said first end is equipped with a cap, the cap in communication with a handle;
wherein said second end is equipped with a conical tip;
wherein said conical tip is equipped with bristles;
wherein said bristles are arranged in a spiral pattern, extending clockwise from said brush forming a cone-tipped cylinder;
removing the brush from the opening via the handle, brushing the bristles along the brush-off point to remove excess organic virgin coconut oil;
gripping the handle with the hand;
dragging the bristles of the brush along the follicles of the target area along the grain of the follicles, evenly distributing the organic virgin coconut oil to the target area; and
replacing the brush into the container.

2. The method of claim 1, further comprising grabbing said handle to manually apply the solution to the target area with high dexterity.

3. The method of claim 1, wherein said handle is equipped with a contoured grip.
